(19) United States
(12) Patent Application Publication
(10) Pub. No.: US 2011/0245651 A1
(43) Pub. Date: Oct. 6, 2011

(54) MEDICAL IMAGE PLAYBACK DEVICE AND METHOD, AS WELL AS PROGRAM

(75) Inventor: Keigo NAKAMURA, Tokyo (JP)

(73) Assignee: FUJIFILM CORPORATION, Tokyo (JP)

(21) Appl. No.: 13/017,222

(22) Filed: Jan. 31, 2011

(30) Foreign Application Priority Data
Mar. 31, 2010 (JP) 2010-080170

(51) Int. Cl. A61B 5/05

(52) U.S. Cl. 600/407

(57) ABSTRACT

Time-series images of a heart and sound data representing heart sounds are obtained. The time-series images of the heart is temporally synchronized with the sound data representing the heart sounds, and the time-series images of the heart and the sound data representing the heart sounds synchronized with each other are played back.

### BACKGROUND OF THE INVENTION

1. Field of the Invention

The present invention relates to a medical image playback device, a medical image playback method, and a medical image playback program for playing back time-series images of the heart.

2. Description of the Related Art

Along with advancement of medical devices (such as a multi-detector CT apparatus) in recent years, high-quality high-resolution three-dimensional images are beginning to be used for imaging diagnosis. The three-dimensional image is formed by a number of two-dimensional images and thus has a large amount of information. Therefore, it may take time for a doctor to find and diagnose a desired observation part. To address this problem, it has been practiced to enhance visibility of an entire organ or a lesion to improve efficiency of diagnosis by recognizing an organ of interest, and extracting the organ of interest from a three-dimensional image containing the organ of interest with using a method, such as maximum intensity projection (MIP) and minimum intensity projection (MinIP), to achieve MIP display, to achieve volume rendering (VR) display of the three-dimensional image, or to achieve CPR (Curved Planer Reconstruction) display.

Further, with respect to medical devices for obtaining three-dimensional images, those capable of high-speed imaging have been developed, and this enables obtaining three-dimensional images in chronological order in short time intervals. Therefore, by performing time-series display of an organ of interest contained in such three-dimensional images obtained in chronological order, i.e., by performing four-dimensional display, which means three dimensions plus time, observation of movement of the organ of interest can be achieved in such a manner that one observes a moving image (see U.S. Pat. No. 7,339,587, which will hereinafter be referred to as Patent Document 1).

In particular, by taking three-dimensional images of the heart in chronological order, and displaying the heart extracted from the three-dimensional image in chronological order, movement of the heart along with heartbeat can be displayed as a high-resolution three-dimensional moving image, and this enables to check abnormality (if any) in the heart movement.

In the case where there exists a heart disease, the heart sounds include murmur, and therefore the heart sounds provide useful information for diagnosing the heart movement. To this end, various techniques have been proposed for analyzing a heart disease by analyzing heart sound data. For example, in U.S. Pat. No. 6,477,405 (hereinafter, Patent Document 2), a technique to determine an opening time of the aortic valve by carrying out frequency analysis of the heart sound data is proposed. Further, in U.S. Pat. No. 6,824,519 (hereinafter, Patent Document 3), a technique to determine a heart sound component based on a frequency bandwidth, which is found by carrying out frequency analysis of the heart sound data, is proposed. Still further, in Japanese Unexamined Patent Publication No. 2002-153434 (hereinafter, Patent Document 4), a technique to analyze a heart disease by carrying out frequency analysis of the heart sound data is proposed. Yet further, in Japanese Unexamined Patent Publication No. 2009-240527 (hereinafter, Patent Document 5), a technique involving carrying out frequency analysis of the heart sound data, and analyzing a heart disease based on a frequency with the maximum signal intensity in spectrum power density data of the heart sound data for one heart sound period, signal intensity thresholds set in the spectrum power density data and a frequency width relative to each threshold is proposed.

With the technique disclosed in Patent Document 1, however, although check of the heart movement can be achieved, check of the heart sounds cannot be achieved. Further, with the techniques disclosed in Patent Documents 2 to 5, although check of a heart disease can be achieved by analyzing the heart sounds, check of the heart movement cannot be achieved.

### SUMMARY OF THE INVENTION

In view of the above-described circumstances, the present invention is directed to enabling diagnosis of heart movement using heart sounds in combination.

An aspect of the medical image playback device according to the invention includes:

image obtaining means for obtaining time-series images of a heart;

sound obtaining means for obtaining sound data representing heart sounds;

synchronizing means for temporally synchronizing the time-series images of the heart with the sound data representing the heart sounds; and

playback control means for playing back the time-series images of the heart and the sound data representing the heart sounds synchronized with each other.

The “time-series images of the heart” may be any images, as long as the heart movement can be played back by displaying the images in chronological order. Specific examples thereof include three-dimensional images of the heart extracted from three-dimensional images, two-dimensional images containing the heart at a certain slice position of three-dimensional images, or images of the heart obtained by simple X-ray imaging. Besides the time-series images of the heart itself, time-series images of a structure forming the heart, such as the coronary artery, may be used.

In the medical image playback device according to the invention, the synchronizing means may temporally synchronize the time-series images of the heart with the sound data representing the heart sounds based on a reference electrocardiographic waveform.

In the medical image playback device according to the invention, the playback control means may be capable of adjusting a playback time of the time-series images of the heart and the sound data representing the heart sounds synchronized with each other.

In the medical image playback device according to the invention, the time-series images of the heart and the sound data representing the heart sounds may be obtained at the same time.

In the medical image playback device according to the invention, the time-series images of the heart and the sound data representing the heart sounds may be obtained at different times.

The “different times” may be times temporally apart from each other. It may be preferred that the sound data representing the heart sounds is obtained at a time before or after the time when the time-series images of the heart are obtained, and that the state of the subject when the sound data is obtained is the same as that when the time-series images of the heart are obtained. In this case, the subject is in the resting state with lying on an imaging bed during imaging, and the sound data representing the heart sounds may be obtained from the subject in the resting state with lying on the imaging bed before or after the imaging.

An aspect of the medical image playback method according to the invention includes:

obtaining time-series images of a heart;

obtaining sound data representing heart sounds;

temporally synchronizing the time-series images of the heart with the sound data representing the heart sounds; and

playing back the time-series images of the heart and the sound data representing the heart sounds synchronized with each other.

The medical image playback method according to the invention may be provided in the form of a program for causing a computer to carry out the medical image playback method.

According to the invention, the time-series images of the heart and the sound data representing the heart sounds are obtained, the time-series images of the heart is temporally synchronized with the sound data representing the heart sounds, and the time-series images of the heart and the sound data representing the heart sounds synchronized with each other are played back. Thus, the heart sounds can be played back synchronously with the heartbeat, thereby enabling accurate diagnosis of the heart using the heart movement and the heart sounds.

Further, by temporally synchronizing the time-series images of the heart with the sound data representing the heart sounds based on a reference electrocardiographic waveform, accurate synchronization of the heartbeat with the heart sounds can be achieved.

Still further, by enabling adjustment of the playback time of the time-series images of the heart and the sound data representing the heart sounds synchronized with each other, the time-series images of the heart and the sound data representing the heart sounds can be played back at a different playback speed depending on the adjusted playback time.

Yet further, by obtaining the time-series images of the heart and the sound data representing the heart sounds at the same time, the heart movement is aligned with the heart sounds, thereby achieving more accurate diagnosis of the heart using the heart movement and the heart sounds.

Further, even when it is difficult to obtain the sound data representing the heart sounds at the same time when the time-series images of the heart is obtained, substantial alignment between the heart movement and the heart sounds can be achieved by obtaining the time-series images of the heart and the sound data representing the heart sounds at different times under the same situation, such as when the subject is in the resting state. As a result, relatively accurate diagnosis of the heart using the heart movement and the heart sounds can be achieved.

### BRIEF DESCRIPTION OF THE DRAWINGS

FIG. 1 is a schematic block diagram illustrating the configuration of a medical image playback device according to an embodiment of the invention,

FIG. 2 is a diagram illustrating an electrocardiographic waveform,

FIG. 3 is a diagram illustrating an electrocardiographic waveform, a heart sound waveform and a heart murmur waveform being synchronized with each other,

FIG. 4 is a flow chart illustrating a process carried out in the embodiment,

FIG. 5 is a diagram illustrating three-dimensional volume data of a heart played back on a display in chronological order,

FIG. 6 is a diagram illustrating a window used to adjust a playback time, and

FIG. 7 is a diagram illustrating a state where three-dimensional volume data of a heart and an ultrasonographic image are displayed.

### DESCRIPTION OF THE PREFERRED EMBODIMENTS

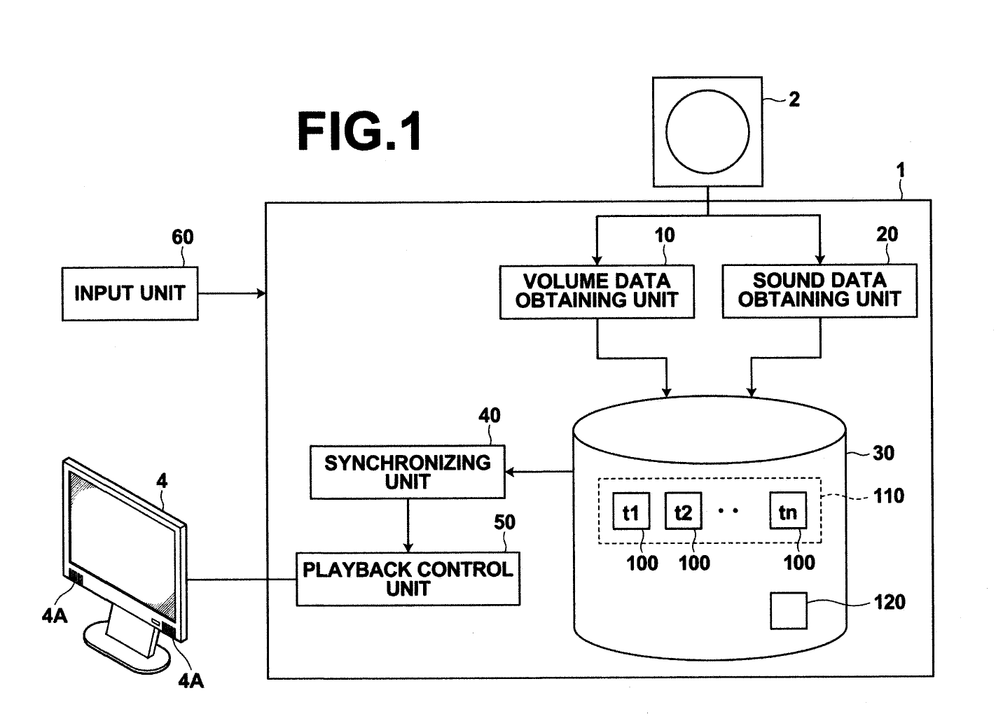

Hereinafter, an embodiment of the present invention will be described with reference to the drawings. FIG. 1 is a schematic block diagram illustrating the configuration of a medical image playback device according to the embodiment of the invention. It should be noted that the configuration of the medical image playback device 1 shown in FIG. 1 is implemented by executing a medical image playback processing program, which is read in an auxiliary storage device of a computer, on the computer. The medical image playback processing program is stored in a storage medium, such as a CD-ROM, or is distributed over a network, such as the Internet, to be installed on the computer.

The medical image playback device 1 according to this embodiment includes a volume data obtaining unit 10, a sound data obtaining unit 20, a storage unit 30, a synchronizing unit 40, a playback control unit 50 and an input unit 60.

The volume data obtaining unit 10 has a function of a communication interface, which obtains a three-dimensional volume data group 110 formed by pieces of three-dimensional volume data 100 obtained by imaging the heart of a subject at a modality 2, such as a CT apparatus or an MRI apparatus, in predetermined time intervals Δt. It should be noted that the three-dimensional volume data group 110 is sent from the modality 2 via a LAN.

The three-dimensional volume data 100 is obtained by arranging pieces of two-dimensional tomographic image data in layers. The pieces of two-dimensional tomographic image data are sequentially obtained along a direction perpendicular to a slice plane of the heart, which is the object of diagnosis. In this embodiment, the three-dimensional volume data 100 is generated by arranging tomographic images, which are taken with the modality 2, such as a CT apparatus or an MRI apparatus, in layers. It should be noted that the volume data obtained with a CT apparatus is data storing an X-ray absorption for each voxel, where each voxel is assigned with a single pixel value (if imaging is carried out using a CT apparatus, a value representing the X-ray absorption).

The three-dimensional volume data group 110 includes, for example, a series of three-dimensional volume data 100, which are obtained by imaging the subject in certain time intervals Δt, i.e., at a time t1, a time t2, . . . , and a time tn.

It should be noted that the three-dimensional volume data 100 has associated information, which is defined by the DICOM (Digital Imaging and Communications in Medicine) standard, added thereto. The associated information may contain, for example, an image ID for identifying a three-dimensional image represented by each three-dimensional volume data 100, a patient ID for identifying each subject, an examination ID for identifying each examination, a unique ID (UID) assigned to each image information, an examination date and time when each image information is generated, a type of modality used in an examination to obtain each image information, patient information, such as name, age, sex, etc., of each patient, examined part (imaged part, which is the heart in this embodiment), imaging conditions (such as whether or not a contrast agent is used, a radiation dose, etc.), a serial number or a collection number assigned to each image when a plurality of images are obtained in a single examination, etc.

Further, the three-dimensional volume data 100 obtained in this embodiment is obtained by imaging the heart of the subject. Therefore, the associated information contains information of an RR interval. Now, the RR interval is described.

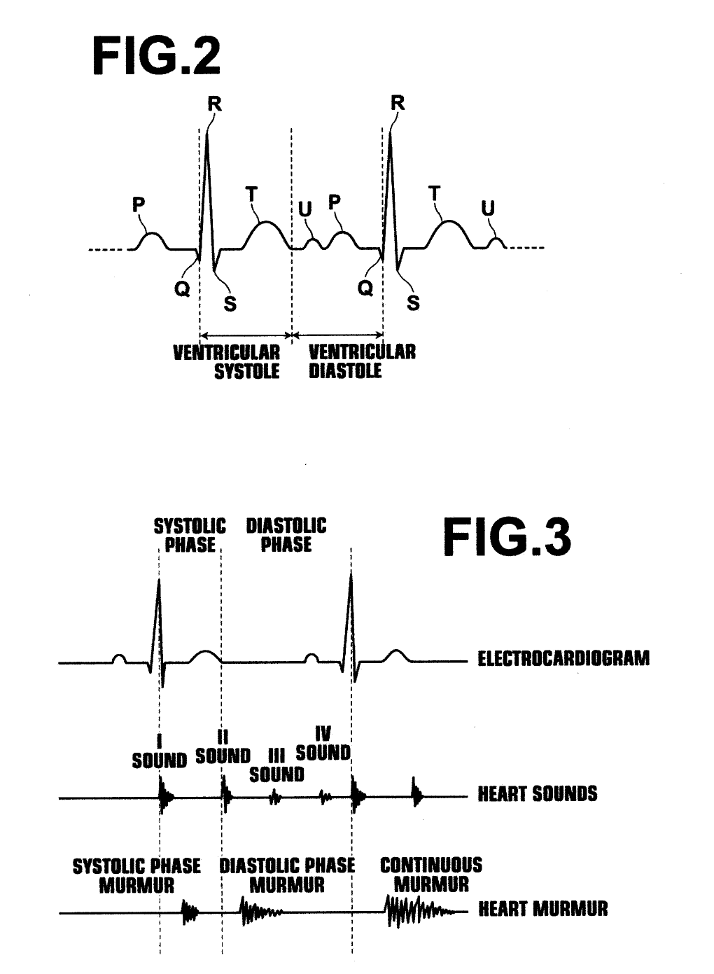

FIG. 2 is a diagram illustrating an electrocardiographic waveform. As shown in FIG. 2, the electrocardiographic waveform includes P waves, Q waves, R waves, S waves, T waves and U waves, where the R waves exhibit the largest amplitude. The period between the Q wave and the end of the T wave indicates the ventricular systole, and the period between the end of the T wave and the next Q wave indicates the ventricular diastole. The RR interval is indicative of timing, at which the image represented by the three-dimensional volume data having the RR interval information was obtained, with a percentage value indicating a point between an R wave and the next R wave in the electrocardiogram. For example, an RR interval value of 0% indicates that the three-dimensional volume data 100, which has the associated information containing that RR interval, was obtained at timing corresponding to the start of the R wave. An RR interval value of 50% indicates that the three-dimensional volume data 100, which has the associated information containing that RR interval, was obtained at timing corresponding to a point that divides the period between the R waves in half.

The sound data obtaining unit 20 has a function of a communication interface to obtain sound data 120 representing heart sounds during imaging, which is detected from the subject with using a microphone or a sensor, for example, attached at a predetermined position on the chest of the subject during imaging of the subject at the modality 2. The sound data 120 is sent from the modality 2 via the LAN. It should be noted that the sound data representing the heart sounds is not limited to one that is obtained from the subject simultaneously with the imaging. For example, if it is difficult to obtain the heart sounds from the subject during imaging, the sound data may be obtained from the subject before or after the imaging with the subject being in the same state as a state of the subject during the imaging. Alternatively, the sound data may be an artificially generated heart sound. The heart sounds are not limited to those obtained using a microphone, and may be obtained using other means, such as a stethoscope.

The storage unit 30 is a large-capacity storage device, such as a hard disk, and stores the three-dimensional volume data group 110 and the sound data 120.

The synchronizing unit 40 temporally synchronizes the three-dimensional volume data group 110 of the heart with the sound data 120 representing the heart sounds. Now, the synchronization is described. As described above, the associated information of the DICOM standard added to each three-dimensional volume data 100 contains the RR interval. Therefore, the synchronizing unit 40 obtains the information of the RR interval contained in the associated information of each three-dimensional volume data 100, and aligns the RR interval of each three-dimensional volume data 100 with a certain electrocardiographic waveform which is used as a reference (reference electrocardiographic waveform) to synchronize the three-dimensional volume data 100 with the reference electrocardiographic waveform.

The heart movement includes a diastolic phase and a systolic phase, where the maximum dilatation of the heart is achieved when the left ventricle is in its smallest state. It is known that the maximum dilatation state of the heart is achieved when the RR interval value is 70 to 80%. Therefore, the synchronizing unit 40 may synchronize the three-dimensional volume data 100 with the reference electrocardiographic waveform by extracting the heart from the three-dimensional volume data 100, extracting the three-dimensional volume data 100 containing the heart in the maximum dilatation state from the three-dimensional volume data group 110, aligning the extracted three-dimensional volume data 100 with a position corresponding to 70 to 80% of the RR interval in the reference electrocardiographic waveform, and using this position as a reference to align the remaining pieces of the three-dimensional volume data 100 with the reference electrocardiographic waveform.

Next, synchronization of the sound data 120 is described. FIG. 3 illustrates an electrocardiographic waveform, a heart sound waveform and a heart murmur waveform being synchronized with each other. As shown in FIG. 3, the heart sound waveform includes I sounds, II sounds, III sounds and IV sounds, and the heart murmur includes systolic phase murmur, diastolic phase murmur and continuous murmur. Therefore, the sound data 120 representing the heart sounds is synchronized with the reference electrocardiographic waveform by obtaining the heart sound waveform by applying signal processing to the sound data, and aligning the sound data 120 with the reference electrocardiographic waveform such that the start position of the I sound in the heart sound waveform is aligned with the peak position of the R wave in the reference electrocardiographic waveform.

Further, as can be seen from FIG. 3, the position in the heart murmur where the diastolic phase murmur ends corresponds to the RR interval value of 70 to 80%. Therefore, synchronizing unit 40 may synchronize the sound data 120 with the reference electrocardiographic waveform by aligning the sound data 120 with the reference electrocardiographic waveform such that the end position of the diastolic phase heart murmur is aligned with the position corresponding to the RR interval value of 70 to 80% in the reference electrocardiographic waveform.

In this manner, the three-dimensional volume data group 110 and the sound data 120 are synchronized with the reference electrocardiographic waveform to achieve the temporal synchronization of the three-dimensional volume data group 110 and the sound data 120.

It should be noted that the synchronization at the synchronizing unit 40 may be carried out when the three-dimensional volume data group 110 is played back, or the synchronization may be carried out when the device 1 has obtained the three-dimensional volume data group 110 and the sound data 120. In the latter case, the three-dimensional volume data group 110 and the sound data 120 temporally synchronized with each other may be stored in the storage unit 30. Further, the three-dimensional volume data group 110 and the sound data 120 temporally synchronized with each other may be combined and converted into moving image data of the MPEG format, or the like, to be stored in the storage unit 30.

The playback control unit 50 plays back the three-dimensional volume data group 110 and the sound data 120 temporally synchronized with each other on a display 4 provided with a speaker 4A when an instruction to play back the three-dimensional volume data group 110 is fed to the device 1 via the input unit 60. It should be noted that the three-dimensional volume data group 110 may be played back in a manner of MIP display, VR display or CPR display, for example, after heart extraction or coronary artery extraction has been carried out. Further, a two-dimensional image representing a cross section of the heart obtained along the same slice plane in each three-dimensional volume data 100 may be extracted from the three-dimensional volume data 100, and the extracted two-dimensional images may be played back in chronological order.

The heart extraction may be achieved using a technique that involves carrying out part recognition, as disclosed, for example, in U.S. Patent Application Publication No. 20080267481, and extracting the heart based on a result of the part recognition. The technique disclosed in U.S. Patent Application Publication No. 20080267481 involves normalizing inputted tomographic images forming the three-dimensional volume data 100, calculating a number of feature quantities from the normalized tomographic images, inputting the feature quantities calculated for each normalized tomographic image to a classifier, which is obtained by using an AdaBoost technique, to calculate, for each part, a score indicating a likelihood of being the part, and determining the part (i.e., the heart) shown in each tomographic image based on the calculated part scores using dynamic programming so that the order of the body parts of a human body is maintained. Further, a method using template matching (see, for example, Japanese Unexamined Patent Publication No. 2002-253539) or a method using eigenimages of each part (i.e., the heart) (see, for example, U.S. Pat. No. 7,245,747) may be used.

The heart extraction may also be achieved using a technique disclosed in Japanese Unexamined Patent Publication No. 2009-211138. In the technique disclosed in Japanese Unexamined Patent Publication No. 2009-211138, first, a tomographic image represented by two-dimensional tomographic image data forming the three-dimensional volume data 100 is displayed, and the user sets an arbitrary point in a heart region in the tomographic image using a pointing device, or the like (the set point will hereinafter be referred to as a user setting point). Then, with using a classifier obtained through machine learning, such as the AdaBoost technique, corners of the contour of the heart region are detected as reference points. Further, for each point (voxel) in a three-dimensional area having a size large enough to contain the heart (which will hereinafter be referred to as an area to be processed) with the user setting point being the center, an evaluation value indicating whether or not the point is a point on the heart contour is calculated using a classifier obtained through machine learning, such as the AdaBoost technique. Points on the periphery of the area to be processed are determined in advance to be points in a background area out of the heart region, and the user setting point and the reference points are determined in advance to be points in the heart region. Then, using the evaluation values of the points in the area to be processed, the heart region is extracted from the three-dimensional image represented by the three-dimensional volume data 100 by applying a graph cutting method.

The coronary artery extraction may be achieved using methods proposed, for example, in Japanese Patent Application Nos. 2009-048679 and 2009-069895. These methods extract the coronary artery as follows. First, positions of candidate points forming a core line of the coronary artery and a major axis direction are calculated based on values of the voxel data forming the volume data. Alternatively, positional information of candidate points forming the core line of the coronary artery and the major axis direction may be calculated by calculating a Hessian matrix with respect to the volume data, and analyzing eigenvalues of the calculated Hessian matrix. Then, a feature quantity representing a likelihood of being the coronary artery is calculated for each voxel data around the candidate points, and whether or not the voxel data represents the coronary artery region is determined based on the calculated feature quantity. The determination based on the feature quantity is carried out based on an evaluation function, which is obtained in advance through machine learning. In this manner, the extraction of the coronary artery of the heart from the three-dimensional volume data 100 is achieved.

The input unit 60 is formed by a known input device, such as a keyboard and a mouse.

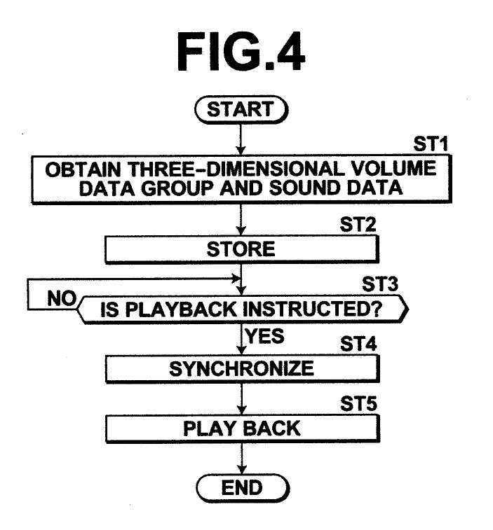

Next, a process carried out in this embodiment is described. FIG. 4 is a flow chart illustrating the process carried out in this embodiment. First, the volume data obtaining unit 10 and the sound data obtaining unit 20 obtain the three-dimensional volume data group 110 of the heart and the sound data 120 representing the heart sounds from the modality 2, as described above (step ST1), and the obtained data are stored in the storage unit 30 (step ST2). When an instruction to play back the three-dimensional volume data group 110 is fed via the input unit 60 (step ST3: YES), the synchronizing unit 40 synchronizes the three-dimensional volume data group 110 with the reference electrocardiographic waveform and synchronizes the sound data 120 with the reference electrocardiographic waveform to achieve synchronization of the three-dimensional volume data group 110 with the sound data 120 (step ST4). Then, the playback control unit 50 plays back the three-dimensional volume data group 110 of the heart and the sound data 120 representing the heart sounds synchronized with each other on the display 4 (step ST5), and the process ends.

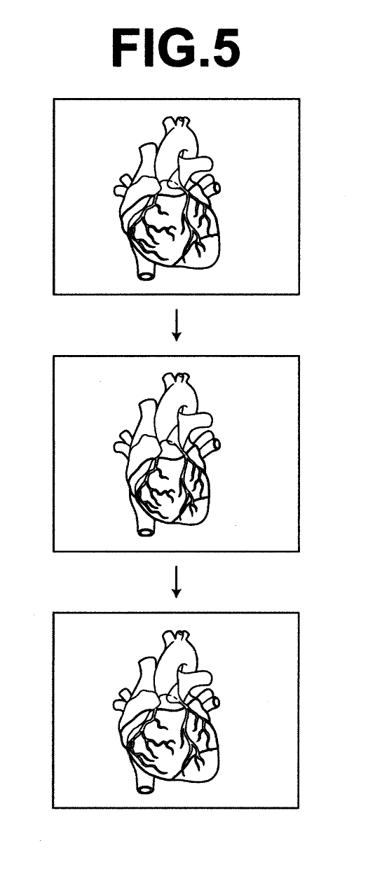

FIG. 5 is a diagram illustrating the three-dimensional volume data 100 of the heart played back on the display 4 in chronological order. As shown in FIG. 5, VR display, for example, of states of the heartbeat is shown on the display 4, and the heart sounds are played back on the speaker 4A synchronously with the heartbeat. In the case where the coronary artery is extracted, states of the coronary artery can be played back synchronously with the heartbeat together with the heart sounds.

As described above, in this embodiment, the three-dimensional volume data group 110 of the heart and the sound data 120 representing the heart sounds are obtained, the three-dimensional volume data group 110 is temporally synchronized with the sound data 120, and the three-dimensional volume data group 110 of the heart and the sound data 120 representing the heart sounds synchronized with each other are played back. Thus, the heart sounds can be played back synchronously with the heartbeat, thereby enabling accurate diagnosis of the heart using the heart movement and the heart sounds.

Further, since the three-dimensional volume data group 110 of the heart is temporally synchronized with the sound data 120 representing the heart sounds based on the reference electrocardiographic waveform, accurate synchronization of the heartbeat with the heart sounds can be achieved.

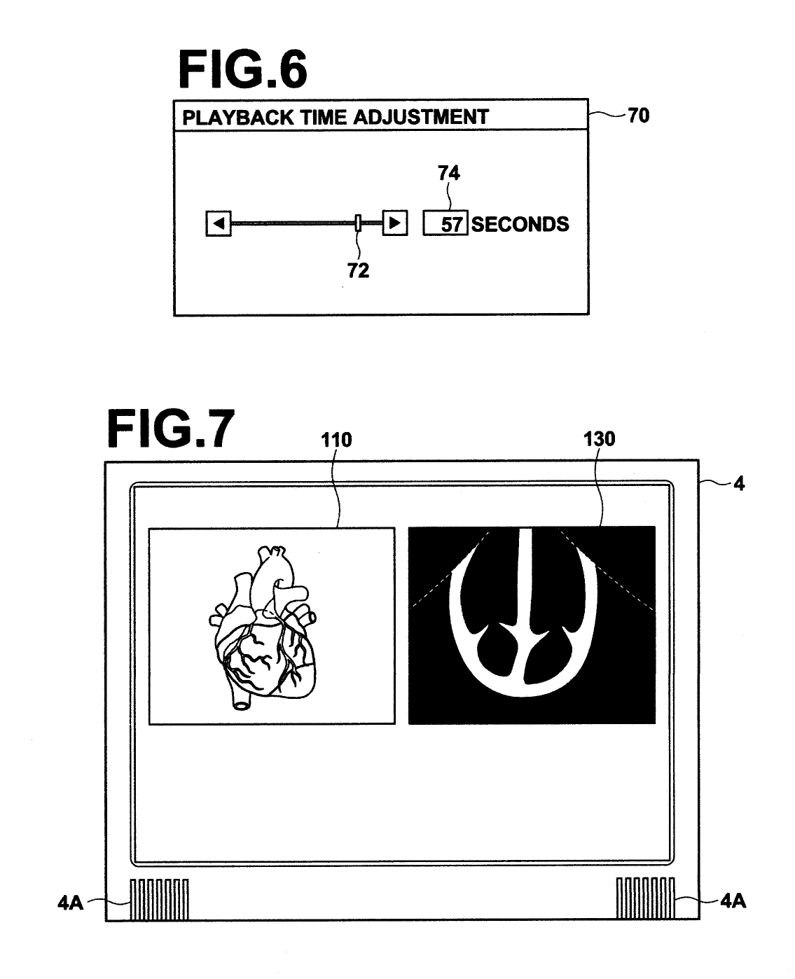

It should be noted that, in the above-described embodiment, a playback time for playing back the three-dimensional volume data group 110 and the sound data 120 may be changed. Specifically, as shown in FIG. 6, a window 70 for adjustment of the playback time may be displayed on the display 4, and the operator may control an adjustment tab 72 in the window 70 to adjust the playback time displayed in a time display area 74. When the playback time is decreased, the playback speed increases. When the playback time is increased, the playback speed decreases.

Although the case where the three-dimensional volume data group 110 of the heart is played back in chronological order has been described in the above-described embodiment, the time-series images of the heart are not limited to the three-dimensional volume data 100, and may be an image group formed by a series of images of the heart obtained by simple X-ray imaging carried out in predetermined time intervals.

Further, although the heart sounds are obtained using a microphone, or the like, in the above-described embodiment, the sound data representing the heart sounds of the subject may be obtained using an ultrasonic diagnosis device. In this case, an ultrasonographic signal of the heart is obtained, and a Doppler signal representing blood flow information is detected based on the ultrasonographic signal. The Doppler signal is converted into the sound data representing the heart sounds, and the sound data obtaining unit 20 obtains this sound data. In such a state where the valve of the heart is not completely closed, the blood flows through a gap of the valve and thus flows at a very high flow rate. At this time, the sound data converted from the Doppler signal represents a high note. In the reverse situation, the sound data represents a low note. Therefore, by aligning an interval between a high note and the next high note (or an interval between a low note and the next low note) in the sound data with the RR interval in the reference electrocardiographic waveform, synchronization of the sound data with the reference electrocardiographic waveform can be achieved.

It should be noted that, in the case where the sound data representing the heart sounds is obtained using an ultrasonic diagnosis device, an ultrasonographic image of the heart can simultaneously be obtained. In this case, an ultrasonographic image 130 of the heart can be synchronously played back on the display 4 in addition to the three-dimensional volume data group 110 of the heart, as shown in FIG. 7. This enables comprehensive diagnosis using the three-dimensional volume data of the heart, the ultrasonographic image of the heart and the heart sounds.

What is claimed is:

1. A medical image playback device comprising:
image obtaining means for obtaining time-series images of a heart;
sound obtaining means for obtaining sound data representing heart sounds;
synchronizing means for temporally synchronizing the time-series images of the heart with the sound data representing the heart sounds; and
playback control means for playing back the time-series images of the heart and the sound data representing the heart sounds synchronized with each other.

2. The medical image playback device as claimed in claim 1, wherein the synchronizing means temporally synchronizes the time-series images of the heart with the sound data representing the heart sounds based on a reference electrocardiographic waveform.

3. The medical image playback device as claimed in claim 1, wherein the playback control means is capable of adjusting a playback time of the time-series images of the heart and the sound data representing the heart sounds synchronized with each other.

4. The medical image playback device as claimed in claim 1, wherein the time-series images of the heart and the sound data representing the heart sounds are obtained at the same time.

5. The medical image playback device as claimed in claim 1, wherein the time-series images of the heart and the sound data representing the heart sounds are obtained at different times.

6. The medical image playback device as claimed in claim 1, wherein the time-series images of the heart comprise three-dimensional images obtained by imaging the heart in predetermined time intervals.

7. The medical image playback device as claimed in claim 6, wherein the three-dimensional images are taken with a CT apparatus or an MRI apparatus.

8. A medical image playback method comprising:
obtaining time-series images of a heart;
obtaining sound data representing heart sounds;
temporally synchronizing the time-series images of the heart with the sound data representing the heart sounds; and
playing back the time-series images of the heart and the sound data representing the heart sounds synchronized with each other.

9. A computer-readable recording medium containing a medical image playback program for causing a computer to carry out the steps of:
obtaining time-series images of a heart;
obtaining sound data representing heart sounds;
temporally synchronizing the time-series images of the heart with the sound data representing the heart sounds; and
playing back the time-series images of the heart and the sound data representing the heart sounds synchronized with each other.
